(19) United States
(12) Patent Application Publication
(10) Pub. No.: US 2026/0076691 A1
(43) Pub. Date: Mar. 19, 2026

(54) MEDICAL DEVICE AND METHOD FOR MANUFACTURING MEDICAL DEVICE

(71) Applicant: ASAHI INTECC CO., LTD., Seto-shi (JP)

(72) Inventor: Kenta TSUGE, Seto-shi (JP)

(73) Assignee: ASAHI INTECC CO., LTD., Seto-shi (JP)

(21) Appl. No.: 19/330,360

(22) Filed: Sep. 16, 2025

(30) Foreign Application Priority Data
Sep. 18, 2024 (JP) 2024-160691

(51) Int. Cl. A61B 17/22; A61B 17/00; A61M 25/09

(52) U.S. Cl. CPC A61B 17/22; A61B 17/00234; A61M 25/09; A61B 2017/00238; A61B 2017/00292; A61B 2017/00526; A61B 2017/22042; A61B 2017/22094; A61M 2025/09083; A61M 2025/09108; A61M 2025/09175; A61M 2207/00

(57) ABSTRACT

A medical device includes a main body part and a leading portion that is connected to a distal end of the main body part and is configured to enter a lesion, the main body part including a coil portion of a wire rod in which a part of the wire rod is spirally wound, and the leading portion including a loop portion of the wire rod that extends beyond a distal end of the coil portion of the wire rod toward a distal end side of the medical device.

### CROSS REFERENCE TO RELATED APPLICATIONS

This application claims the benefit of JP 2024-160691, filed Sep. 18, 2024. The disclosure of the prior application is hereby incorporated by reference herein in its entirety.

### TECHNICAL FIELD

The technology disclosed herein relates to a medical device and a method for manufacturing the medical device.

### BACKGROUND ART

Guide wires are used to treat stenosis sites or occlusion sites (hereinafter referred to as lesion) in blood vessels. Known guide wires include a loop portion (see, for example, Patent Document 1).

### PRIOR ART DOCUMENT

### Patent Document

Patent Document 1: Japanese Translation of PCT International Publication 2006-507899

### SUMMARY

### Technical Problem

Known guide wires leave room for improvement in strength.

This specification discloses a technology capable of solving the above-described issue.

### Solution to Problem

A medical device disclosed herein includes a main body part and a leading portion that is connected to a distal end of the main body part and configured to enter a lesion, the main body part including a coil portion of a wire rod in which the wire rod is spirally wound, and the leading portion including a loop portion of the wire rod that extends beyond a distal end of the coil portion of the wire rod toward a distal end side of the medical device.

### BRIEF DESCRIPTION OF DRAWINGS

FIG. 1 is a plan view of a guide wire according to a first embodiment.

FIG. 2 is a side view of the guide wire according to the first embodiment.

FIG. 3 is a sectional view of the guide wire according to the first embodiment taken along the line III-III of FIG. 2.

FIG. 4 is a partially enlarged plan view illustrating a loop portion and a portion in the vicinity thereof in a coil member according to the first embodiment in an enlarged manner.

FIG. 5 is a flowchart illustrating an example of a method for manufacturing the guide wire according to the first embodiment.

FIG. 6 is an explanatory view illustrating an example of the method for manufacturing the guide wire according to the first embodiment.

FIG. 7 is an explanatory view illustrating an example of the method for manufacturing the guide wire according to the first embodiment.

FIG. 8 is an explanatory view illustrating an example of a treatment method using the guide wire according to the first embodiment.

FIG. 9 is an explanatory view illustrating an example of the treatment method using the guide wire according to the first embodiment.

FIG. 10 is a partially enlarged plan view illustrating a leading portion and a portion in the vicinity thereof in a guide wire according to a second embodiment in an enlarged manner.

FIG. 11 is a partially enlarged sectional view illustrating a leading portion and a portion in the vicinity thereof in a guide wire according to a third embodiment, which is cut at the same position as the line III-III in FIG. 2.

FIG. 12 is a partially enlarged plan view illustrating a leading portion and a portion in the vicinity thereof in a guide wire according to a fourth embodiment in an enlarged manner.

FIG. 13 is a partially enlarged plan view illustrating a leading portion and a portion in the vicinity thereof in a guide wire according to a fifth embodiment in an enlarged manner.

FIG. 14 is a partially enlarged side view illustrating the leading portion and the portion in the vicinity thereof in the guide wire according to the fifth embodiment in an enlarged manner.

FIG. 15 is a partially enlarged plan view illustrating a leading portion and a portion in the vicinity thereof in a guide wire according to a sixth embodiment in an enlarged manner.

FIG. 16 is a partially enlarged side view illustrating the leading portion and the portion in the vicinity thereof in the guide wire according to the sixth embodiment in an enlarged manner.

FIG. 17 is a partially enlarged plan view illustrating a leading portion and a portion in the vicinity thereof in a guide wire according to a seventh embodiment in an enlarged manner.

FIG. 18 is a partially enlarged side view illustrating the leading portion and the portion in the vicinity thereof in the guide wire according to the seventh embodiment in an enlarged manner.

FIG. 19 is a partially enlarged plan view illustrating a leading portion and a portion in the vicinity thereof in a guide wire according to an eighth embodiment in an enlarged manner.

### DESCRIPTION OF EMBODIMENTS

### First Embodiment

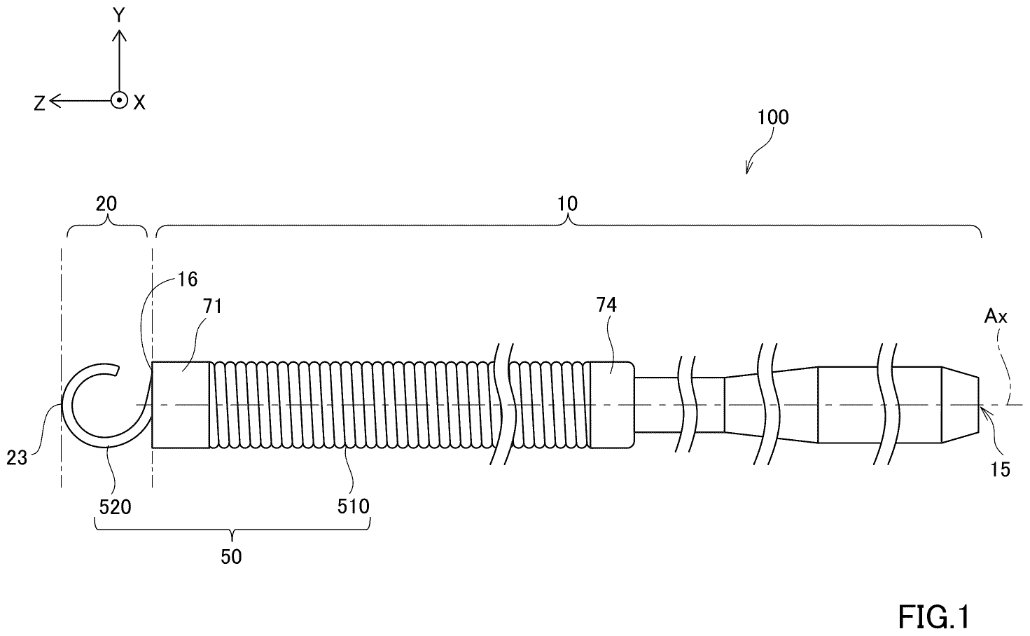

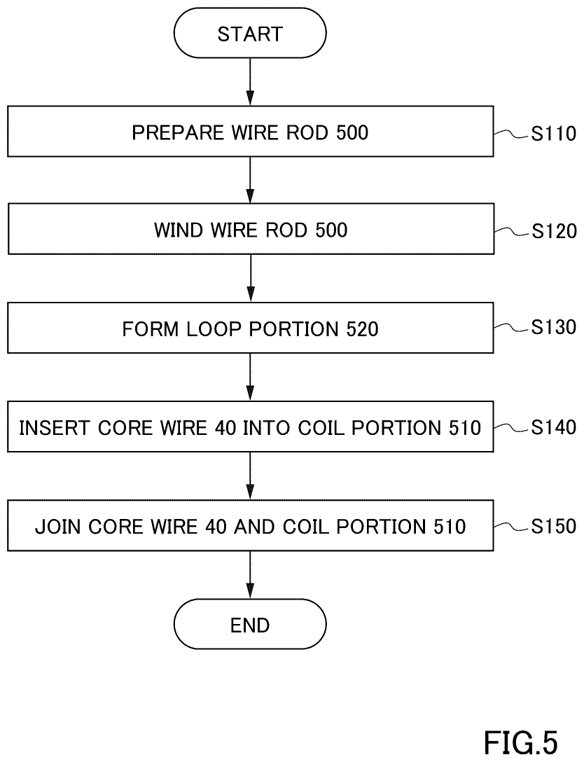

A first embodiment will be described with reference to FIGS. 1 to 9. A guide wire 100 according to the present embodiment is a medical device that is inserted into a living body lumen to treat a lesion in the living body lumen. The living body lumen includes a tubular organ of a human body such as a blood vessel, a digestive tract, a ureter, an organ, or a bile duct. In the guide wire 100, the Z-axis positive direction side is the distal end side to be inserted into a body, and the Z-axis negative direction side is a proximal end side to be operated by a professional such as a doctor. The distal end side is also referred to as a far side, and the proximal end side is also referred to as a near side. In each drawing, a part of the guide wire 100 may be omitted. FIGS. 1, 2, and 3 illustrate the guide wire 100 in a straight state parallel to the Z-axis. The guide wire 100 is flexible enough to be curved. The same applies to the subsequent drawings.

In this specification, with regard to the guide wire 100 and each constituent member thereof, an end on the distal end side is referred to as a “distal end”, the distal end and the vicinity thereof are referred to as a “distal end portion”, an end on the proximal end side is referred to as a “proximal end”, and the proximal end and the vicinity thereof are referred to as a “proximal end portion”. The transverse cross-section of the guide wire 100 and each constituent member thereof means a cross-section perpendicular to the longitudinal direction. The longitudinal cross-section of the guide wire 100 and each constituent component thereof means a cross-section parallel to the center axis in the longitudinal direction. In the guide wire 100 and each constituent component thereof, a direction perpendicular to the longitudinal direction is referred to as a radial direction. The outer diameter of the guide wire 100 and each constituent component thereof means a width in the radial direction.

The guide wire 100 is a medical device to be inserted into a living body lumen such as a blood vessel. The entire length of the guide wire 100 is, for example, 1000 mm or more and 3000 mm or less.

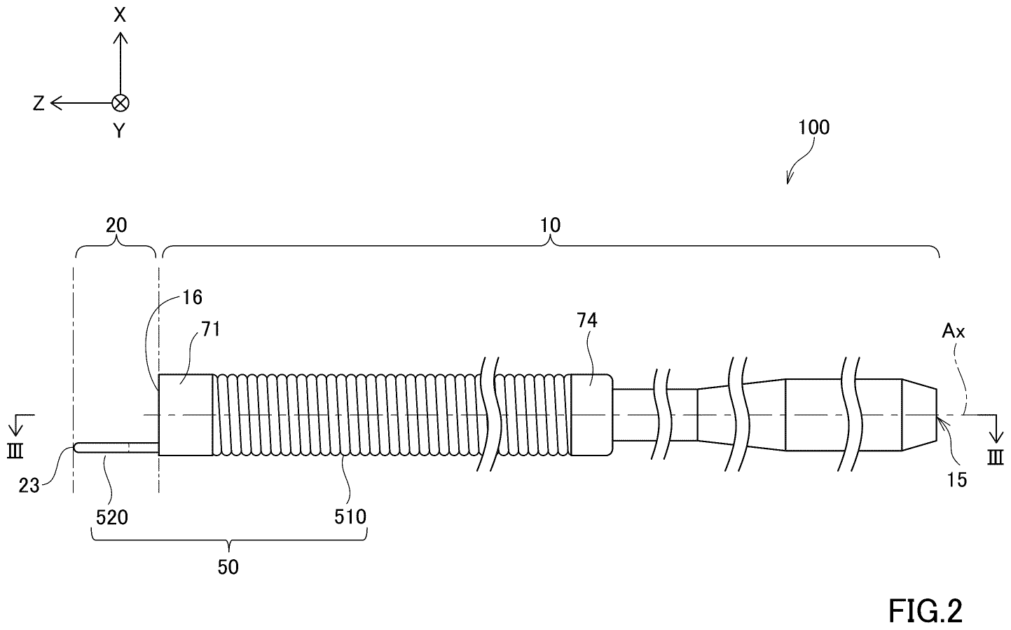

As illustrated in FIGS. 1 and 2, the guide wire 100 includes a main body part 10 and a leading portion 20.

The main body part 10 is an elongated portion extending along a center axis Ax. According to the present embodiment, the center axis Ax of the main body part 10 coincides with the center axis of the guide wire 100. A proximal end 15 of the main body part 10 coincides with the proximal end of the guide wire 100.

The leading portion 20 may be referred to as a lead portion, a drill portion, a crushing portion, a peeling portion, an entry portion, a peeler, a shaver, or the like. A distal end 23 of the leading portion 20 coincides with the distal end of the guide wire 100. The surface of the leading portion 20 may have an edge or may have no edge. An edge is a boundary between two surfaces. The leading portion 20 enters a lesion while rotating around the center axis Ax. Entering the lesion by the leading portion 20 may be expressed as crossing or passing through the lesion, drilling the lesion, crushing the lesion, peeling the lesion, raking the lesion, going under the lesion, getting into the lesion, or the like. A length L20 of the leading portion 20 along the center axis Ax is, for example, 0.2 mm or more and 2.0 mm or less. The length L20 of the leading portion 20 may be 0.3 mm or more and 1.5 mm or less, or may be 0.4 mm or more and 1.0 mm or less.

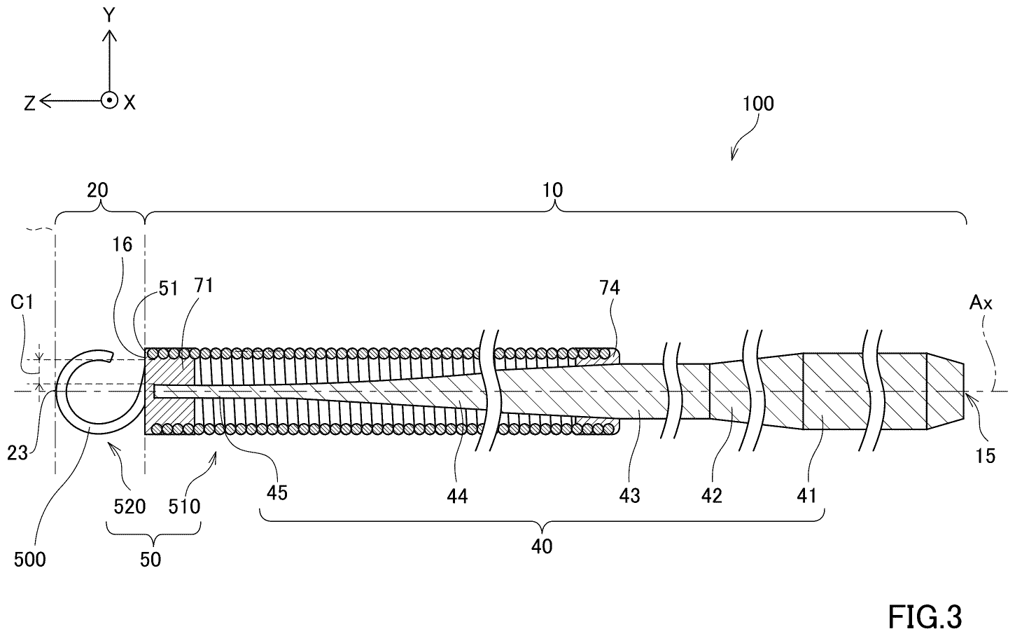

As illustrated in FIG. 3, the guide wire 100 includes a core wire 40 and a coil member 50.

The core wire 40 is a rod-shaped member. The core wire 40 includes a large diameter portion 41, a first tapered portion 42, an intermediate diameter portion 43, a second tapered portion 44, and a small diameter portion 45. The large diameter portion 41, the first tapered portion 42, the intermediate diameter portion 43, the second tapered portion 44, and the small diameter portion 45 are continuous in this order from the proximal end of the core wire 40.

The large diameter portion 41 is a rod-shaped portion having a substantially constant outer diameter. The outer diameter of the large diameter portion 41 is, for example, about 0.2 mm to 3.0 mm. The intermediate diameter portion 43 is a rod-shaped portion that is located closer to the distal end side than the large diameter portion 41 and has a substantially constant outer diameter smaller than the outer diameter of the large diameter portion 41. The first tapered portion 42 is a portion that is located between the large diameter portion 41 and the intermediate diameter portion 43 and has a diameter gradually decreasing from the boundary with the large diameter portion 41 toward the boundary with the intermediate diameter portion 43. The small diameter portion 45 is a rod-shaped portion that is located closer to the distal end side than the intermediate diameter portion 43 and has a substantially constant outer diameter smaller than the outer diameter of the intermediate diameter portion 43. The second tapered portion 44 is a portion that is located between the intermediate diameter portion 43 and the small diameter portion 45 and has a diameter gradually decreasing from the boundary with the intermediate diameter portion 43 toward the boundary with the small diameter portion 45.

The shape of the transverse cross-section of the core wire 40 at each position may be any shape. The shape of the transverse cross-section of the core wire 40 at each position may be a circle, a partial circle, an ellipse, a rectangle, a parallelogram, a trapezoid, a rhombus, or the like. A partial circle is one of the two halves of a circle divided by a chord. The outer edge of the partial circle includes an arc line and a line segment connecting two end portions of the arc line. The partial circle may have, for example, a semicircular shape, a partially circular shape, or an arcuate shape. A semicircular shape is one of two halves of a circle divided by a chord passing through the center of the circle. A partially circular shape is the larger of two halves of a circle divided by a chord that does not pass through the center of the circle. An arcuate shape is the smaller of the two halves divided by a chord that does not pass through the center of the circle. The transverse cross-section of the core wire 40 is not strictly limited to the above-described shapes, and may be substantially the above-described shapes. The shape of the transverse cross-section may be different at each position along the longitudinal direction of the core wire 40.

The material of the core wire 40 is, for example, metal. To be more specific, the material of the core wire 40 may be, for example, stainless steels such as SUS302, SUS304, and SUS316, Ni—Ti alloys, and piano wires. The entire core wire 40 may be formed of the same material, or each portion of the core wire 40 may be formed of a different material.

The coil member 50 is formed of a single wire rod 500. The wire rod forming the coil member 50 may be a single strand or a twisted strand formed by twisting a plurality of strands.

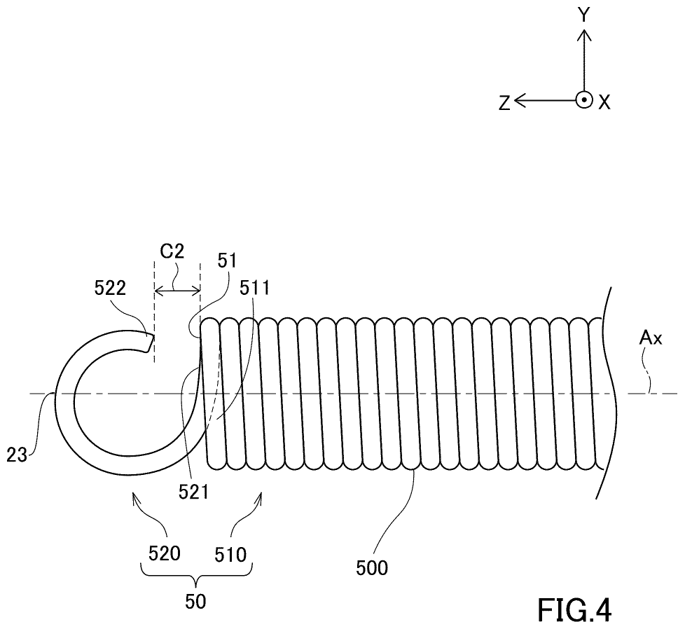

As illustrated in FIGS. 3 and 4, the coil member 50 includes a coil portion 510 and a loop portion 520. The coil portion 510 is a hollow cylindrical portion in which most of the wire rod 500 except for the distal end portion is spirally wound. The coil portion 510 surrounds a part of the core wire 40. According to the present embodiment, the second tapered portion 44 and the small diameter portion 45 of the core wire 40 are covered with the coil portion 510. The second tapered portion 44 and the small diameter portion 45 are separated from the coil portion 510. That is, there is a clearance C1 between the second tapered portion 44 and the coil portion 510 and between the small diameter portion 45 and the coil portion 510.

The outer diameter of the coil portion 510 is, for example, 0.1 mm or more and 0.6 mm or less. The outer diameter of the coil portion 510 may be 0.2 mm or more and 0.5 mm or less, or may be 0.3 mm or more and 0.4 mm or less. The outer diameter of the coil portion 510 may be 1.00 mm or more and 2.00 mm or less, may be 1.10 mm or more and 1.65 mm or less, or may be 1.20 mm or more and 1.35 mm or less.

According to the present embodiment, the outer diameter of the coil portion 510 is constant over the entire length of the coil portion 510. The coil portion 510 may have a tapered shape in which the outer diameter gradually decreases from the proximal end toward the distal end, or may have a tapered shape in which the outer diameter gradually decreases from the distal end toward the proximal end. A distal end 51 of the coil portion 510 substantially coincides with a distal end 16 of the main body part 10.

The loop portion 520 is a loop-shaped portion formed by a portion of the wire rod 500 closer to the distal end side than the coil portion 510. The loop portion 520 extends at an angle relative to one turn 511 of the coil portion 510 at the most distal end side. The loop portion 520 includes the distal end 23 of the leading portion 20. That is, the loop portion 520 extends from the distal end 51 of the coil portion 510 to the distal end 23 of the leading portion 20 while being curved, is folded back at the distal end 23, and extends to the vicinity of the distal end 51 of the coil portion 510 while being curved. According to the present embodiment, the loop portion 520 has a substantially circular shape having an outer diameter substantially equal to the outer diameter of the coil portion 510. In this specification, the term “loop shape” includes not only the case where the loop portion has a closed ring, but also the case where there is a clearance between both ends of the loop portion or the case where both ends of the loop portion intersect each other. According to the present embodiment, the loop portion 520 includes a base portion 521 that is a boundary position with the coil portion 510 and a first end portion 522 opposite to the base portion 521. The base portion 521 is a portion of the loop portion 520 provided closer to the distal end side than the distal end 51 of the coil portion 510 and is a portion adjacent to the distal end 51 of the coil portion 510. The first end portion 522 is a terminal portion of the wire rod 500. There is a clearance between the base portion 521 and the first end portion 522. The first end portion 522 and the coil portion 510 are separated from each other. That is, there is a clearance C2 between the first end portion 522 and the coil portion 510. The first end portion 522 is located closer to the distal end side than the coil portion 510.

The material of the coil member 50 is, for example, metal. The material of the coil member 50 may be a material that transmits radiation or a material that does not transmit radiation. The material that transmits radiation may be, for example, stainless steels such as SUS302, SUS304, and SUS316, Ni—Ti alloys, and piano wires. The material that does not transmit radiation may be, for example, platinum, gold, tungsten, or an alloy of any of these. The entire coil member 50 may be formed of the same material, or each portion of the coil member 50 may be formed of a different material.

The distal end portion of the coil portion 510 is joined to the core wire 40 by a distal end side joining member 71. The distal end side joining member 71 enters the inside of the coil portion 510 and joins a part of the small diameter portion 45 and the distal end portion of the coil portion 510. A proximal end portion of the coil portion 510 is joined to the core wire 40 by a proximal end side joining member 74. The coil portion 510 may be joined to the core wire 40 via a joining member formed at another position. The material of the distal end side joining member 71 and the proximal end side joining member 74 is, for example, solder, a brazing material, or an adhesive. The solder may be, for example, an Au—Sn alloy, an Sn—Ag alloy, an Sn—Pb alloy, or a Pb—Ag alloy. The brazing material may be, for example, an aluminum alloy brazing material, a silver brazing material, or a gold brazing material. The adhesive may be, for example, an epoxy-based adhesive.

The main body part 10 includes the core wire 40, the coil portion 510, the distal end side joining member 71, and the proximal end side joining member 74. The leading portion 20 includes the loop portion 520. Next, an example of a method for manufacturing the guide wire 100 will be described.

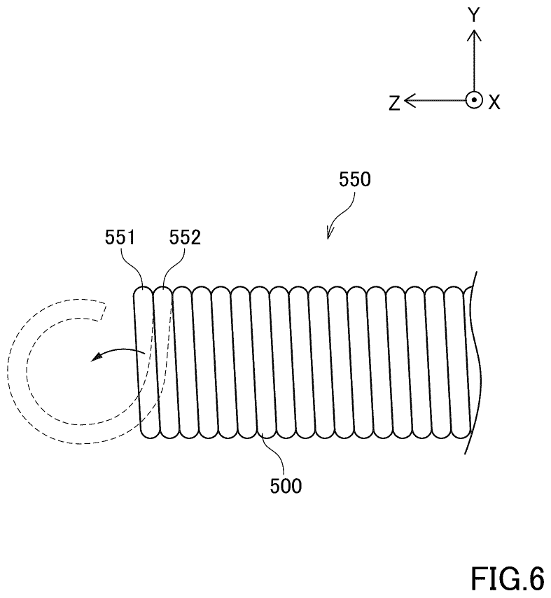

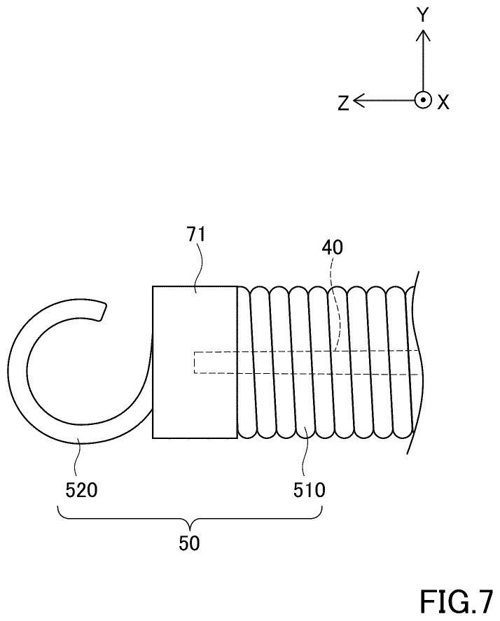

First, the wire rod 500 serving as the material of the coil member 50 is prepared (S110). The wire rod 500 is spirally wound to form a material coil 550 (S120, FIG. 6). Subsequently, the distal end portion of the wire rod 500 is formed into a loop shape (S130, FIG. 6). Specifically, the one turn 551 of the material coil 550 located at the most distal end is lifted to form an angle with another one turn 552 located adjacent thereto. Accordingly, the lifted one turn 551 becomes the loop portion 520, and the remaining portion becomes the coil portion 510. Subsequently, the distal end portion of the core wire 40 is inserted into the inside of the coil portion 510 (S140, FIG. 7). Subsequently, the core wire 40 is joined to the coil portion 510 by the distal end side joining member 71 and the proximal end side joining member 74 (S150, FIG. 7). The guide wire 100 according to the present embodiment is manufactured by the above-described process, for example.

Next, an example of a treatment method using a catheter 120 including the guide wire 100 will be described.

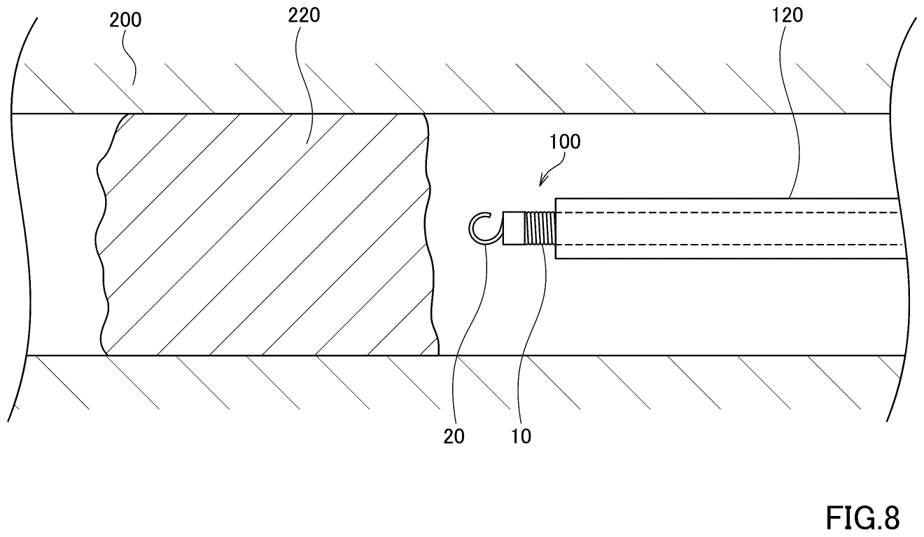

The professional inserts a preceding guide wire (not illustrated) into a blood vessel 200 and advances the preceding guide wire to the front of a lesion 220. The preceding guide wire is referred to as a workhorse guide wire, a first choice guide wire, or the like. Subsequently, the professional inserts the catheter 120 into the blood vessel 200 along the preceding guide wire. The professional advances the catheter 120 to the front of the lesion 220 in the blood vessel 200. Subsequently, the professional removes the preceding guide wire from the blood vessel 200. Then, the professional inserts the guide wire 100 into the catheter 120, which is inserted into the blood vessel 200, with the leading portion 20 at the head (FIG. 8). The professional advances the guide wire 100 to the front of the lesion 220 in the blood vessel 200. When the guide wire 100 is advanced, the guide wire 100 may be rotated around the center axis Ax or may not be rotated.

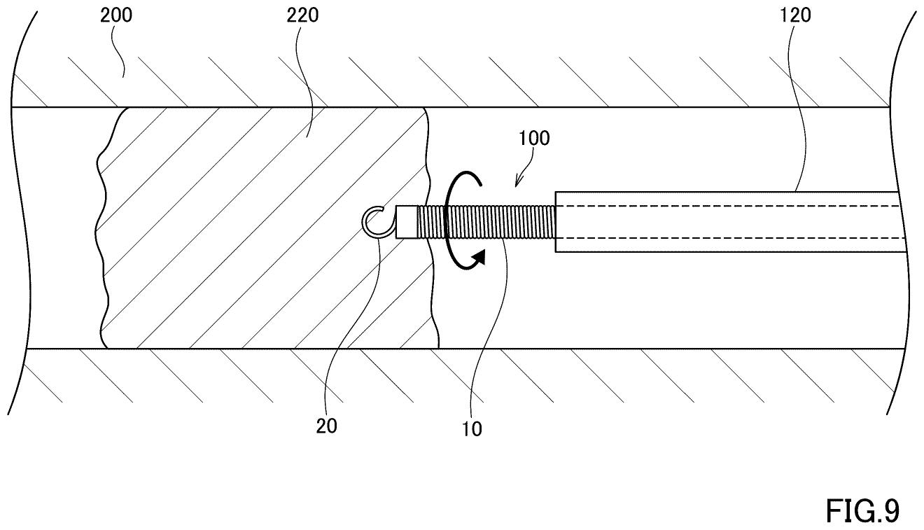

Subsequently, the professional advances the leading portion 20 into the lesion 220 by advancing the guide wire 100 to the distal end side while rotating the guide wire 100 (FIG. 9). When the professional grips the proximal end portion of the guide wire 100 and rotates the guide wire 100 around the center axis Ax, the leading portion 20 located at the distal end portion of the guide wire 100 also rotates around the center axis Ax. The leading portion 20 rotating in the lesion 220 excavates so as to cut the lesion 220.

As described above, the guide wire 100 according to the present embodiment includes the main body part 10 and the leading portion 20 that is connected to the distal end of the main body part 10 and enters the lesion 220. The main body part 10 includes the coil portion 510 in which the wire rod 500 is spirally wound. The leading portion 20 includes the loop portion 520 that is a part of the wire rod 500 and extends beyond the distal end 51 of the coil portion 510 toward the distal end side. This configuration improves the strength of the guide wire 100 since there is no joint between the main body part 10 and the leading portion 20.

According to the present embodiment, the loop portion 520 includes the first end portion 522 including the terminal end of the wire rod 500. The first end portion 522 is located closer to the distal end side than the coil portion 510.

According to the present embodiment, the main body part 10 includes the core wire 40 connected to the coil portion 510. The core wire 40 and the coil portion 510 are separated from each other.

According to the present embodiment, the first end portion 522 and the coil portion 510 are separated from each other.

### Second Embodiment

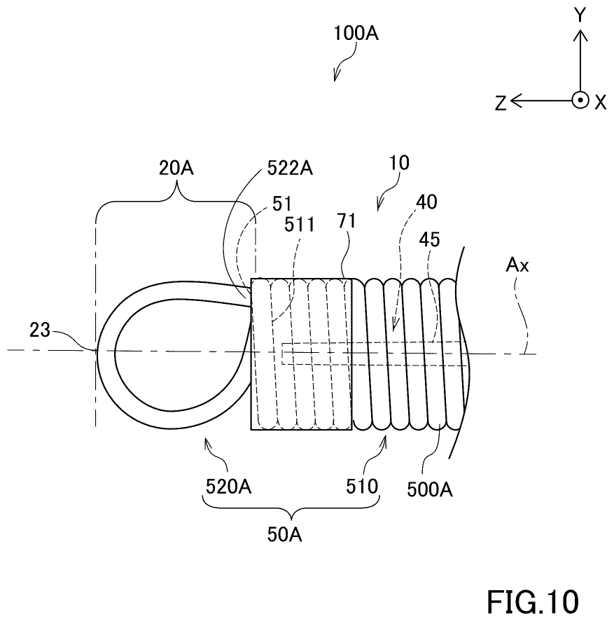

A second embodiment will be described with reference to FIG. 10. In a guide wire 100A according to the present embodiment, a part of the configuration of a coil member 50A is different from that of the first embodiment. According to the present embodiment, the same components as those of the first embodiment are denoted by the same reference numerals, and description thereof will be omitted.

As illustrated in FIG. 10, the guide wire 100A according to the present embodiment includes the core wire 40 and the coil member 50A. As in the first embodiment, the coil member 50A is formed of one wire rod 500A. The coil member 50A has the same configuration as that of the first embodiment except that a first end portion 522A of a loop portion 520A is joined to the distal end 51 of the coil portion 510. A method for joining the first end portion 522A and the coil portion 510 may be, for example, welding or soldering.

As in the first embodiment, the guide wire 100A according to the present embodiment includes the main body part 10 and the leading portion 20A that is connected to the distal end of the main body part 10 and enters the lesion 220. The main body part 10 includes the coil portion 510 in which the wire rod 500A is spirally wound. The leading portion 20A includes the loop portion 520A that is a part of the wire rod 500A and extends beyond the distal end 51 of the coil portion 510 toward the distal end side. This configuration improves the strength of the guide wire 100A.

According to the present embodiment, the first end portion 522A is joined to the distal end 51 of the coil portion 510. This configuration improves the strength of the loop portion 520A.

### Third Embodiment

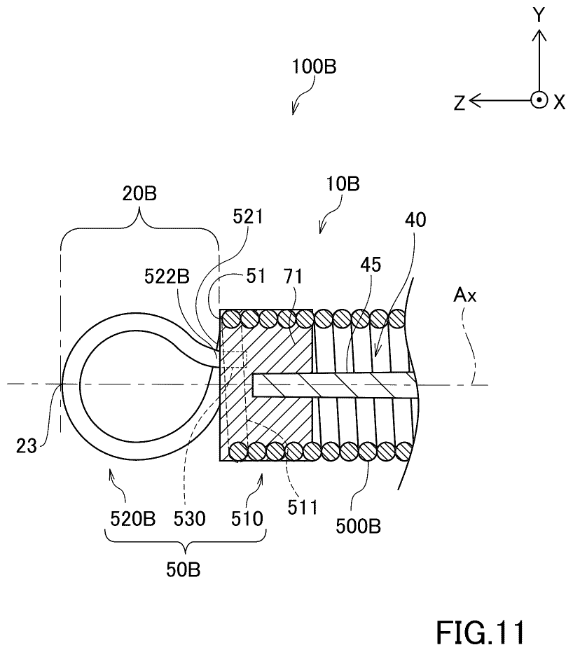

A third embodiment will be described with reference to FIG. 11. In a guide wire 100B according to the present embodiment, a part of the configuration of a coil member 50B is different from that of the first embodiment. According to the present embodiment, the same components as those of the first embodiment are denoted by the same reference numerals, and description thereof will be omitted.

As illustrated in FIG. 11, the guide wire 100B according to the present embodiment includes the core wire 40 and the coil member 50B. As in the first embodiment, the coil member 50B is formed of one wire rod 500B. The coil member 50B includes the coil portion 510, a loop portion 520B, and a second end portion 530. As in the first embodiment, the coil portion 510 is a hollow cylindrical portion in which most of the wire rod 500B except for the distal end portion is spirally wound. The loop portion 520B is a loop-shaped portion formed by a portion of the wire rod 500B closer to the distal end side than the coil portion 510. The loop portion 520B includes a base portion 521 that is a boundary position with the coil portion 510 and a first end portion 522B opposite to the base portion 521. The detailed shape of the loop portion 520B is the same as that of the first embodiment except that the first end portion 522B extends to the distal end 51 of the coil portion 510. The second end portion 530 is a terminal portion of the wire rod 500B extending from the first end portion 522B of the loop portion 520B and is located inside the coil portion 510.

As in the first embodiment, the guide wire 100B according to the present embodiment includes a main body part 10B and a leading portion 20B that is connected to the distal end of the main body part 10B and enters the lesion 220. The main body part 10B includes the coil portion 510 in which the wire rod 500B is spirally wound. The leading portion 20B includes the loop portion 520B that is a part of the wire rod 500B and extends beyond the distal end 51 of the coil portion 510 toward the distal end side. This configuration improves the strength of the guide wire 100B.

According to the present embodiment, the guide wire 100B further includes the second end portion 530 that is continuous with the loop portion 520B and includes the terminal end of the wire rod 500B. The second end portion 530 is located inside the coil portion 510. With this configuration, the second end portion 530 is prevented from being caught on the inner wall of the blood vessel 200. As a result, the safety of the guide wire 100B is enhanced.

### Fourth Embodiment

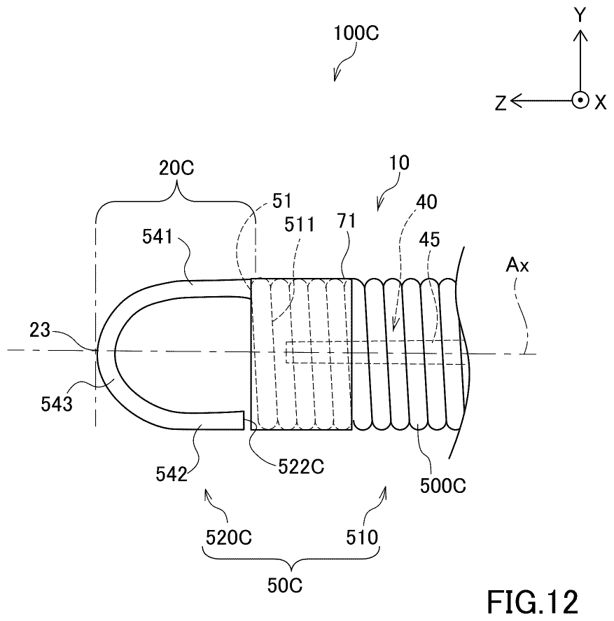

A fourth embodiment will be described with reference to FIG. 12. In a guide wire 100C according to the present embodiment, a part of the configuration of a leading portion 20C is different from that of the first embodiment. According to the present embodiment, the same components as those of the first embodiment are denoted by the same reference numerals, and description thereof will be omitted.

As illustrated in FIG. 12, the guide wire 100C according to the present embodiment includes the core wire 40 and a coil member 50C. As in the first embodiment, the coil member 50C is formed of one wire rod 500C. The coil member 50C includes the coil portion 510 and a loop portion 520C. As in the first embodiment, the coil portion 510 is a hollow cylindrical portion in which most of the wire rod 500C except for the distal end portion is spirally wound. The loop portion 520C is a loop-shaped portion formed by a portion of the wire rod 500C closer to the distal end side than the coil portion 510. According to the present embodiment, the loop portion 520C has a U shape, and a curved portion of the U shape faces the distal end side. That is, the loop portion 520C includes two linear portions 541 and 542, which extend along the center axis Ax and are parallel to each other, and a curved portion 543, which is curved to be convex on the distal end side and connects the distal ends of the two linear portions 541 and 542. The loop portion 520C includes a first end portion 522C including a terminal end of the wire rod 500C. The first end portion 522C is separated from the coil portion 510.

As in the first embodiment, the guide wire 100C according to the present embodiment includes the main body part 10 and the leading portion 20C that is connected to the distal end of the main body part 10 and enters the lesion 220. The main body part 10 includes the coil portion 510 in which the wire rod 500C is spirally wound. The leading portion 20C includes the loop portion 520C that is a part of the wire rod 500C and extends beyond the distal end 51 of the coil portion 510 toward the distal end side. This configuration improves the strength of the guide wire 100C.

### Fifth Embodiment

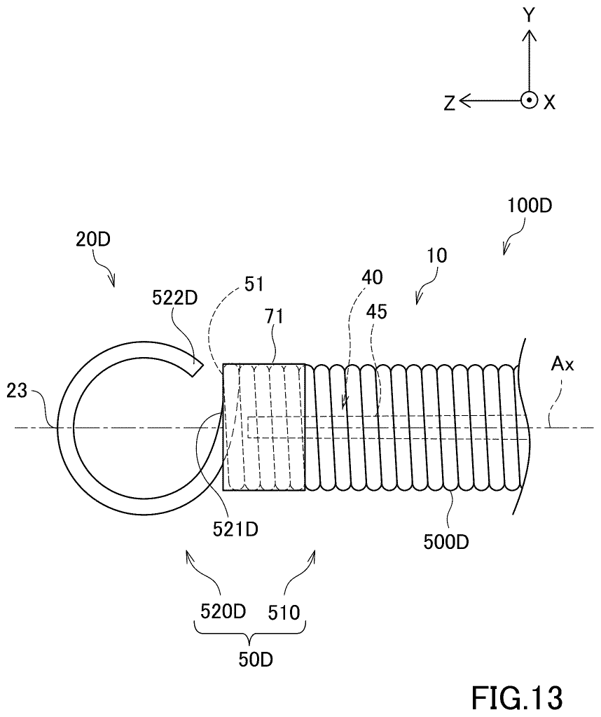

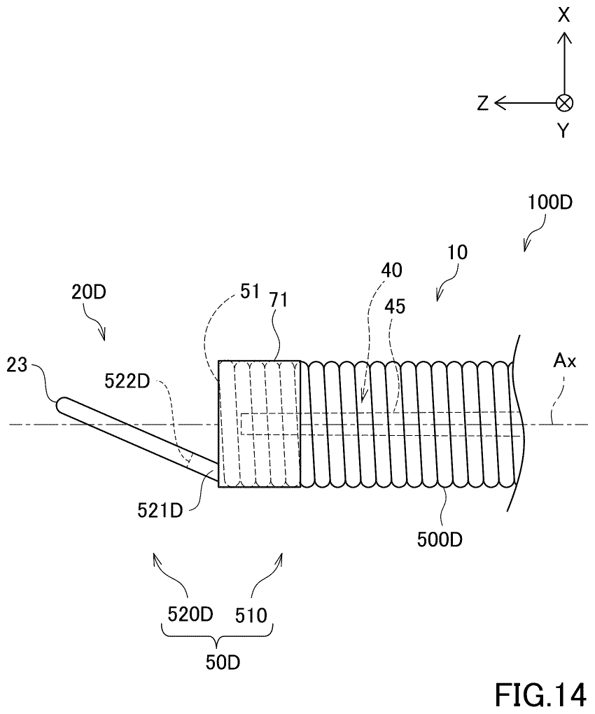

A fifth embodiment will be described with reference to FIGS. 13 and 14. As in the first embodiment, a guide wire 10D according to the present embodiment includes the main body part 10 and a leading portion 20D. In the guide wire 100D according to the present embodiment, a part of the configuration of the leading portion 20D is different from that of the first embodiment. According to the present embodiment, the same components as those of the first embodiment are denoted by the same reference numerals, and description thereof will be omitted.

The guide wire 100D includes the core wire 40 and a coil member 50D. As in the first embodiment, the coil member 50D is formed of one wire rod 500D. The coil member 50D includes the coil portion 510 and a loop portion 520D. The loop portion 520D is a loop-shaped portion formed by a portion of the wire rod 500D closer to the distal end side than the coil portion 510. The loop portion 520D includes a base portion 521D adjacent to the distal end of the coil portion 510 and a first end portion 522D opposite to the base portion 521D. The main body part 10 includes the core wire 40 and the coil portion 510. The leading portion 20D includes the loop portion 520D.

The loop portion 520D includes the distal end 23 of the leading portion 20D. The loop portion 520D extends from the distal end 51 of the coil portion 510 to the distal end 23 of the leading portion 20D while being curved, is folded back at the distal end 23, and extends to the vicinity of the distal end 51 of the coil portion 510 while being curved. According to the present embodiment, the loop portion 520D has a substantially circular shape having an outer diameter one size larger than the outer diameter of the coil portion 510. As illustrated in FIG. 14, the distal end 23 is located closer to the center axis Ax than the base portion 521D. The position of the first end portion 522D and the position of the base portion 521D are different from each other in the direction (X-axis direction) perpendicular to the longitudinal direction (Z-axis direction) of the guide wire 100D. The distance between the center axis Ax and the first end portion 522D in the X-axis direction is shorter than the distance between the center axis Ax and the base portion 521D in the X-axis direction.

As in the first embodiment, the guide wire 100D according to the present embodiment includes the main body part 10 and the leading portion 20D that is connected to the distal end of the main body part 10 and enters the lesion 220. The main body part 10 includes the coil portion 510 in which the wire rod 500D is spirally wound. The leading portion 20D includes the loop portion 520D that is a part of the wire rod 500D and extends beyond the distal end 51 of the coil portion 510 toward the distal end side. This configuration improves the strength of the guide wire 100D.

According to the present embodiment, the main body part 10 extends along the center axis Ax. The loop portion 520D includes the base portion 521D adjacent to the distal end 51 of the coil portion 510. The loop portion 520D includes the distal end 23 of the leading portion 20D. The distal end 23 is located closer to the center axis Ax than the base portion 521D.

### Sixth Embodiment

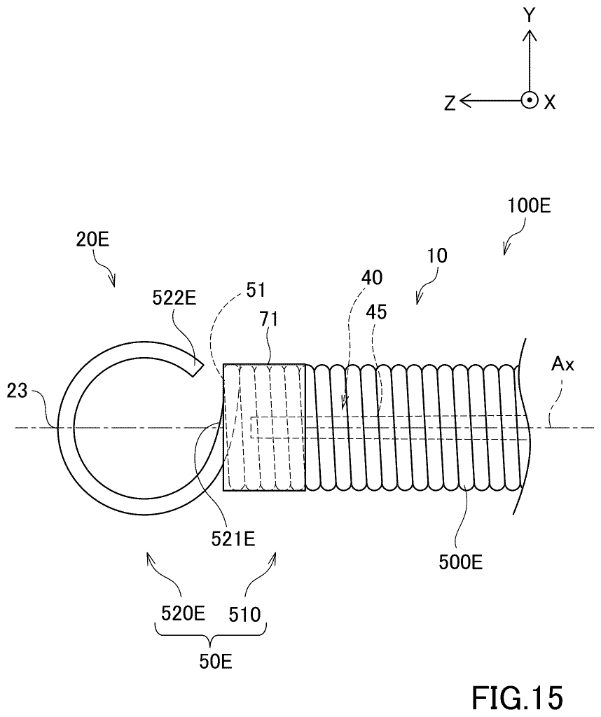

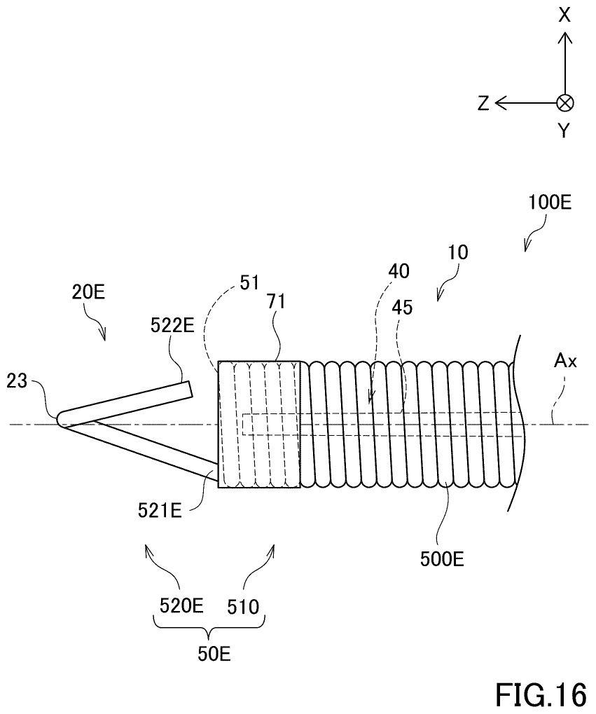

A sixth embodiment will be described with reference to FIGS. 15 and 16. As in the first embodiment, a guide wire 10E according to the present embodiment includes the main body part 10 and a leading portion 20E. The guide wire 100E according to the present embodiment is the same as that of the fifth embodiment except that a loop portion 520E is twisted in a spiral fashion.

The guide wire 100E includes the core wire 40 and a coil member 50E. As in the first embodiment, the coil member 50E is formed of one wire rod 500E. The coil member 50E includes the coil portion 510 and a loop portion 520E. The loop portion 520E has a shape in which a ring one size larger than the outer diameter of the coil portion 510 is twisted in a spiral fashion. More specifically, the loop portion 520E includes a base portion 521E adjacent to the distal end 51 of the coil portion 510 and a first end portion 522E opposite to the base portion 521E. The loop portion 520E includes the distal end 23 of the leading portion 20E. As illustrated in FIG. 16, a portion of the loop portion 520E from the base portion 521E to the distal end 23 extends while being curved to be farther away from the distal end 51 of the coil portion 510 and closer to the center axis Ax as the portion approaches the distal end 23. A portion of the loop portion 520E from the distal end 23 to the first end portion 522E extends while being curved to be closer to the distal end 51 of the coil portion 510 and be farther away from the center axis Ax as the portion approaches the first end portion 522E. The distal end 23 is located closer to the center axis Ax than the base portion 521E. The position of the first end portion 522E and the position of the base portion 521E are different from each other in the direction (X-axis direction) perpendicular to the longitudinal direction (Z-axis direction) of the guide wire 100E. The distance between the center axis Ax and the first end portion 522E in the X-axis direction is shorter than the distance between the center axis Ax and the base portion 521E in the X-axis direction.

As in the first embodiment, the guide wire 100E according to the present embodiment includes the main body part 10 and the leading portion 20E that is connected to the distal end of the main body part 10 and enters the lesion 220. The main body part 10 includes the coil portion 510 in which the wire rod 500E is spirally wound. The leading portion 20E includes a loop portion 520E that is a part of the wire rod 500E and extends beyond the distal end 51 of the coil portion 510 toward the distal end side. This configuration improves the strength of the guide wire 100E.

According to the present embodiment, the main body part 10 extends along the center axis Ax. The loop portion 520E includes the base portion 521E adjacent to the distal end 51 of the coil portion 510. The loop portion 520E includes the distal end 23 of the leading portion 20E. The distal end 23 is located closer to the center axis Ax than the base portion 521E.

### Seventh Embodiment

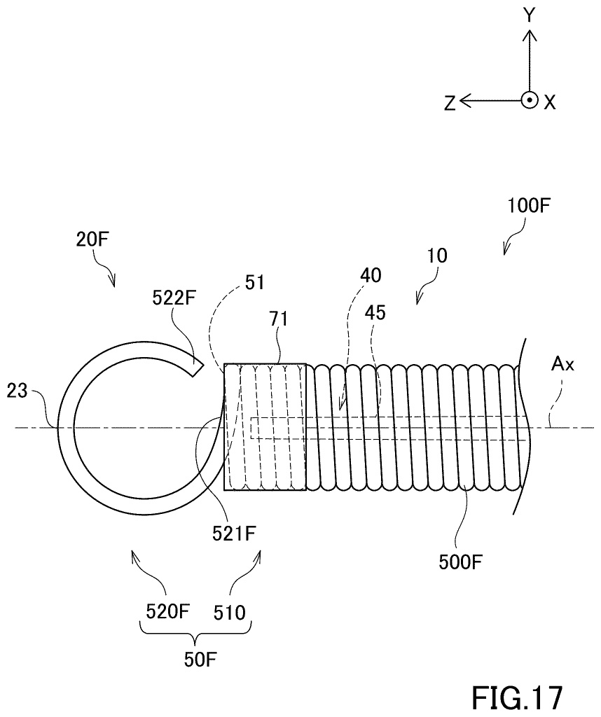

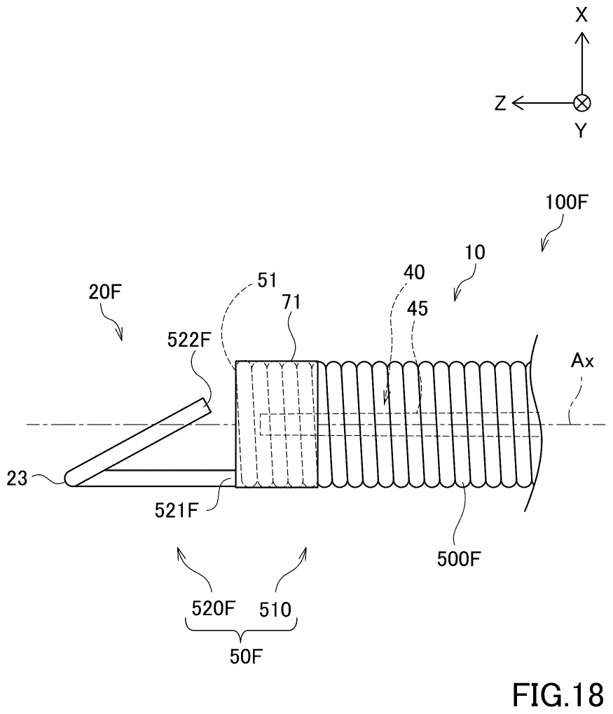

A seventh embodiment will be described with reference to FIGS. 17 and 18. As in the first embodiment, a guide wire 10F according to the present embodiment includes the main body part 10 and a leading portion 20F. The guide wire 100F according to the present embodiment is the same as that of the fifth embodiment except that a part of a loop portion 520F is twisted in a spiral fashion.

The guide wire 100F includes the core wire 40 and a coil member 50F. As in the first embodiment, the coil member 50F is formed of one wire rod 500F. The coil member 50F includes the coil portion 510 and a loop portion 520F. The loop portion 520F has a shape in which a part of a ring one size larger than the outer diameter of the coil portion 510 is twisted in a spiral fashion. More specifically, the loop portion 520F includes a base portion 521F adjacent to the distal end 51 of the coil portion 510 and a first end portion 522F opposite to the base portion 521F. The loop portion 520F includes the distal end 23 of the leading portion 20F. As illustrated in FIG. 18, a portion of the loop portion 520F from the base portion 521F to the distal end 23 extends while being curved to be farther away from the distal end 51 of the coil portion 510 and extend in the direction along the center axis Ax as the portion approaches the distal end 23. A portion of the loop portion 520F from the distal end 23 to the first end portion 522F extends while being curved to be closer to the distal end 51 of the coil portion 510 and be closer to the center axis Ax as the portion approaches the first end portion 522F. The portion of the loop portion 520F from the distal end 23 to the first end portion 522F is located closer to the center axis Ax than the base portion 521F. The position of the first end portion 522F and the position of the base portion 521F are different from each other in the direction (X-axis direction) perpendicular to the longitudinal direction (Z-axis direction) of the guide wire 100F. The distance between the center axis Ax and the first end portion 522F in the X-axis direction is shorter than the distance between the center axis Ax and the base portion 521F in the X-axis direction.

As in the first embodiment, the guide wire 100F according to the present embodiment includes the main body part 10 and the leading portion 20F that is connected to the distal end of the main body part 10 and enters the lesion 220. The main body part 10 includes the coil portion 510 in which the wire rod 500F is spirally wound. The leading portion 20F includes the loop portion 520F that is a part of the wire rod 500F and extends beyond the distal end 51 of the coil portion 510 toward the distal end side. This configuration improves the strength of the guide wire 100F.

According to the present embodiment, the main body part 10 extends along the center axis Ax. The loop portion 520F includes the base portion 521F adjacent to the distal end 51 of the coil portion 510. A part of the loop portion 520F is located closer to the center axis Ax than the base portion 521F.

### Eighth Embodiment

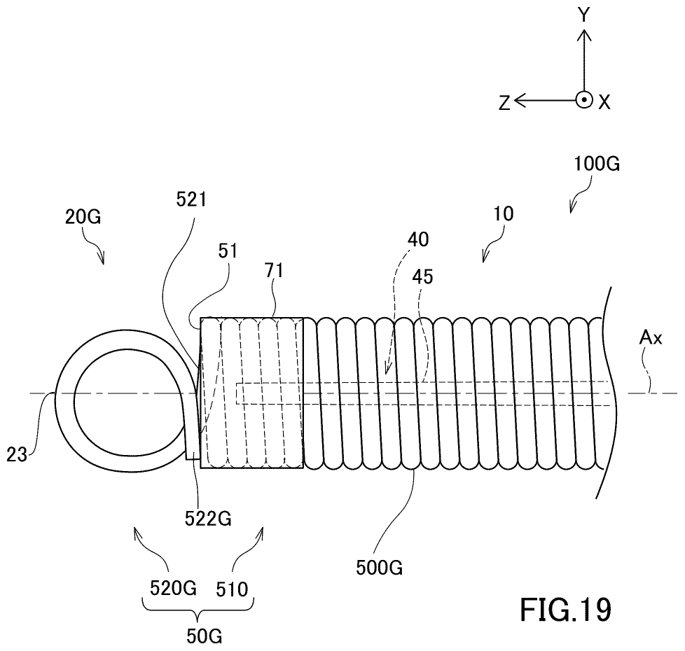

An eighth embodiment will be described with reference to FIG. 19. As in the first embodiment, a guide wire 100G according to the present embodiment includes the main body part 10 and a leading portion 20G. The guide wire 100G according to the present embodiment is different from that of the first embodiment in a part of the configuration of the leading portion 20G. According to the present embodiment, the same components as those of the first embodiment are denoted by the same reference numerals, and description thereof will be omitted.

The guide wire 100G includes the core wire 40 and a coil member 50G. As in the first embodiment, the coil member 50G is formed of one wire rod 500G. The coil member 50G has the same configuration as that of the first embodiment except that a first end portion 522G and the base portion 521 intersect each other.

As in the first embodiment, the guide wire 100G according to the present embodiment includes the main body part 10 and the leading portion 20G that is connected to the distal end of the main body part 10 and enters the lesion 220. The main body part 10 includes the coil portion 510 in which the wire rod 500G is spirally wound. The leading portion 20G includes a loop portion 520G that is a part of the wire rod 500G and extends beyond the distal end 51 of the coil portion 510 toward the distal end side. This configuration improves the strength of the guide wire 100G.

### Modification

The technology disclosed herein is not limited to the above-described embodiments and can be modified into various forms without departing from the gist thereof, and for example, the following modifications are also possible.
(1) The angle of the loop portion with respect to one turn located at the distal end of the coil portion is not limited to that in the above-described embodiment. For example, the loop portion may extend away from the center axis of the coil portion as the loop portion approaches the distal end.
(2) The loop portion may be formed by winding a portion of the wire rod except for the distal end portion in a spiral fashion and then bending the distal end portion.
(3) In the description according to the above embodiment, the guide wire for treating a lesion in a blood vessel is used as an example. The technology disclosed herein is similarly applicable to medical devices in general for treating lesions in living body lumens.

1. A medical device comprising:
a main body part; and
a leading portion that is connected to a distal end of the main body part and is configured to enter a lesion, wherein
the main body part includes a coil portion of a wire rod in which the wire rod is spirally wound, and
the leading portion includes a loop portion of the wire rod that extends beyond a distal end of the coil portion of the wire rod toward a distal end side of the medical device.

2. The medical device according to claim 1, wherein
the loop portion of the wire rod includes a first end portion of the wire rod, and
the first end portion of the wire rod includes a terminal end of the wire rod and is located closer to the distal end side of the medical device than is the coil portion of the wire rod.

3. The medical device according to claim 2, wherein the first end portion of the wire rod and the coil portion of the wire rod are separated from each other.

4. The medical device according to claim 1, wherein
the loop portion of the wire rod includes a first end portion of the wire rod, and
the first end portion of the wire rod includes a terminal end of the wire rod and is joined to the coil portion of the wire rod.

5. The medical device according to claim 1, wherein the loop portion of the wire rod includes a first end portion of the wire rod,
the medical device further comprising a second end portion of the wire rod that is continuous with the loop portion of the wire rod and includes a terminal end of the wire rod, wherein
the first end portion of the wire rod extends to the distal end of the coil portion of the wire rod, and
the second end portion of the wire rod extends from the first end portion of the wire rod and is located inside the coil portion of the wire rod.

6. The medical device according to claim 1, wherein
the main body part includes a core wire connected to the coil portion of the wire rod, and
the core wire and the coil portion of the wire rod are separated from each other.

7. The medical device according to claim 1, wherein
the main body part extends along an axis,
the loop portion of the wire rod includes a base portion adjacent to the distal end of the coil portion of the wire rod, and
at least a part of the loop portion of the wire rod is located closer to the axis than is the base portion.

8. The medical device according to claim 7, wherein
the loop portion includes a distal end of the leading portion, and
the distal end of the leading portion is located closer to the axis than is the base portion.

9. The medical device according to claim 1, wherein
the loop portion of the wire rod includes a first end portion of the wire rod and a base portion adjacent to the distal end of the coil portion, and
the first end portion of the wire rod and the base portion intersect with each other.

10. A method for manufacturing a medical device including
a main body part, and
a leading portion that is connected to a distal end of the main body part and is configured to enter a lesion,
the main body part including a coil portion of a wire rod in which the wire rod is spirally wound, and
the leading portion including a loop portion of the wire rod that extends beyond a distal end of the coil portion of the wire rod toward a distal end side of the medical device,
the method comprising:
preparing the wire rod serving as a material of a coil member comprising the coil portion of the wire rod and the loop portion of the wire rod;
spirally winding the wire rod; and
forming a distal end portion of the wire rod into a loop shape.
